(19) United States
(12) Patent Application Publication
(10) Pub. No.: US 2021/0299657 A1
(43) Pub. Date: Sep. 30, 2021

(54) Method for Development of Microfluidic Assay Device Prototype

(71) Applicant: University of Manitoba, Winnipeg (CA)

(72) Inventors: Francis Lin, Winnipeg (CA); Jiandong Wu, Winnipeg (CA)

(21) Appl. No.: 17/266,204

(22) PCT Filed: Aug. 8, 2019
(86) PCT No.: PCT/CA2019/051085

§ 371 Date: Feb. 5, 2021

Related U.S. Application Data
(60) Provisional application No. 62/722,456, filed on Aug. 24, 2018.
(60) Provisional application No. 62/750,362, filed on Oct. 25, 2018.

(51) Int. Cl. B01L 3/00; G01N 33/50; G01N 33/543

(52) U.S. Cl. CPC B01L 3/502707; B01L 3/502761; G01N 33/5029; G01N 33/54366; B01L 2300/0816; B01L 2200/0694; B01L 2200/12; B01L 2300/12; B01L 2200/0668

(57) ABSTRACT

A method for the preparation of one or more microfluidic chemotactic device prototypes wherein channel and/or barrier dimensions and chemo-attractant and/or cell binding agent concentration and/or type are varied for developing an optimized microfluidic chemotaxis device for a particular cell type and chemo- attractant type as well as instructions for use of same. This process may also require determination of cell density and cell solution volume.

### PRIOR APPLICATION INFORMATION

The instant application claims the benefit of U.S. Provisional Patent Application 62/722,456, filed Aug. 24, 2019 and entitled “Method for optimizing microfluidic assay device prototype”, the entire contents of which are incorporated herein by reference for all purposes.

The instant application also claims the benefit of U.S. Provisional Patent Application 62/750,362, filed Oct. 25, 2019 and entitled “Method for optimizing microfluidic assay device prototype”, the entire contents of which are incorporated herein by reference for all purposes.

### BACKGROUND OF THE INVENTION

Directional cell migration plays an important role in many biological processes and diseases such as host defense, tissue generation and metastatic cancers (1-3). Among the various environmental guiding mechanisms (4, 5), a chemical gradient can direct the migration of different cell types by chemotaxis.

Compared with conventional cell migration assays, microfluidic devices provide useful experimental tools for quantitative cell migration and chemotaxis analysis in well-controlled chemical gradients (7). Various microfluidic gradient devices have been developed and applied to neutrophil chemotaxis analysis (7). In particular, several studies have demonstrated neutrophil migration testing directly from blood by integrating on-chip neutrophil isolation with adhesion-based neutrophil capturing or geometric confinement (6, 8, 9).

### SUMMARY OF THE INVENTION

According to an aspect of the invention, there is provided a method of determining experimental conditions and/or design parameters for a microfluidic cell mobility assay of a particular cell type, said method comprising providing a microfluidic prototype device comprising: a chemical gradient generator; a chemical gradient channel in fluid communication with the chemical gradient generator, said chemical gradient channel arranged to be coated or for coating with a cell binding agent; a cell docking area for receiving a quantity of cells, said cell docking area separated from said chemical gradient channel by a gap channel that is smaller than the average height of a respective one cell of the quantity of cells, said gap channel being formed by a barrier separating the cell docking area and the chemical gradient channel; and micropillars connected from a top of the gap channel to a glass slide, said glass slide for sealing the microfluidic chemotaxis device, said micropillars supporting the gap channel for preventing collapse thereof;

determining depth and width of the chemical gradient channel for generating a suitable, stable gradient of a suitable chemoattractant within the chemical gradient channel;

determining a suitable barrier height for the cell type of interest;

preparing a SU-8 master of a microfluidic device comprising the determined chemical gradient channel depth and the determined chemical gradient channel width and the determined barrier height;

preparing a plurality of PDMS replicas from the PDMS master; and

determining the experimental conditions for the cell mobility assay of the cell type of interest by determining mobility of the cells of the cell type of interest in one of the PDMS replicas while varying at least one of the following parameters:
(1) cell binding molecule applied to the chemical gradient channel;
(2) concentration of the cell binding molecule applied to the chemical gradient channel;
(3) cell density applied to the cell docking area;
(4) sample volume applied to the cell docking area; and
(5) concentration of the chemoattractant in the chemical gradient channel;

and comparing the determined mobilities of individual cells of the cell type of interest under each set of parameters tested to select the parameters for the cell mobility assay using the microfluidic chemotaxis device or microfluidic chemotactic assay unit. In some embodiments, once the experimental conditions are determined, preparing a microfluidic device comprising the optimized chemical gradient channel depth and the optimized chemical gradient channel depth and the optimized barrier height and/or a kit comprising a microfluidic chemotactic device/microfluidic chemotactic assay unit, as well as reagents and instructions for the use thereof.

### BRIEF DESCRIPTION OF THE DRAWINGS

FIG. 1. Flow chart showing steps for fabrication process of the SU-8 master and PDMS device.

FIG. 2. Illustration of the radial microfluidic device and the silicone oil-based pressure-balancing strategy. (A) Design of the whole chip and illustration of a single gradient unit and the cell docking structure. I1 and I2: chemical inlets, O: waste outlet, C: cell loading port; (B) Image shows the real PDMS chip and food dye-colored fluidic networks; (C) Cross sectional illustration of the cell docking structure and micropillar support.

FIG. 3. Illustration of the pressure balancing strategy and validation of gradient generation in the radial microfluidic device. (A) The principle of oil-based pressure balancing strategy. Before balance, one inlet was filled with 15 μL of FITC-dextran medium and the other inlet was filled with 45 μL of medium. The gradient interface was biased to the lower pressure side. After adding one drop of oil to cover and connect these two wells, the gradient interface moves back to the midway of the channel as the pressure is balanced; (B) Image showing the fluorescent gradient of FITC-dextran in the main channel; (C) The gradients in all eight units are identical; (D) The stability of the gradient is shown in one representative gradient unit 6 hours after it had been generated.

FIG. 4. Chemotaxis of neutrophils, MDA-MB-231, and MCF-7 cells in the radial microfluidic device. (A) Representative neutrophil distribution images in the channel at the beginning and at the end of the 20 minutes chemotaxis experiment in a fMLP gradient; (B) Comparison of neutrophil migration distance in a 100 nM IL-8 gradient and a 100 nM fMLP gradient from representative experiments; (C) Representative MDA-MB-231 breast cancer cell distribution images in the channel at the beginning and end of a 6 h migration experiment in different chemical fields, including a medium control, a 100 ng/mL EGF uniform field and a 100 ng/mL EGF gradient; (D) Quantitative migration distance analysis for the experiments in (C); (E) Representative MCF-7 cell distribution images in the channel at the end of a 6 h migration experiment in different chemical fields, including the medium control, a 100 ng/ml EGF uniform field, and a 100 ng/ml EGF gradient; (F) Quantitative results of a migration distance analysis for the experiments in (E). The results are presented as the average value±standard error of the mean (SEM). *indicates p<0.05.

FIG. 5. Cell trajectories, directionality and morphology of MDA-MB-231 in a 100 ng/mL EGF gradient can be deduced in the radial microfluidic device as shown in a representative experiment. (A) Representative final cell distribution and the tracked cell trajectories; (B) Directionality changes over time for three representative individual cells; (C) Directionality distribution in different gradient position intervals (based on the distance from the docking barrier) from a representative experiment. The bottom and top of the red whiskers show the minimum and the maximum value; the red box shows 25%-75% percentile and the middle line in the box shows 50% percentile; the blue square indicates the mean value; (D) The morphology change over time from a representative cell during a 6 h experiment.

FIG. 6. Altered HMGA2 protein expression in MDA-MB-231 breast cancer cells. (A) Comparative Western blot analysis of HMGA2 in MDA-MB-231 cells over-expressing HMGA2 (HMGA2 clone 4) and empty vector control (Mock). C: cytoplasmic; N: nuclear. Lamin A/C and a tubulin were used as nuclear matrix specific and cytoplasmic fraction specific control markers, respectively; (B) Western blot analysis of CRISPR/Cas9 stable MDA-MB-231 clone with targeted homozygous knockout of the HMGA2 gene product. While MDA-MB-231 cells contained endogenous HMGA2 protein, the CRISPR/Cas9 clones were devoid of HMGA2 protein. (C) Treatment with LIN28 inhibitor 1632 small molecule at 10 and 20 μM down-regulated endogenous HMGA2 protein levels in MDA-MB-231 cells after 72 h of treatment. When used at 20 μM, the LIN28 inhibitor 1632 almost completely abolished the presence of HMGA2. NTC stands for non-treatment control.

FIG. 7. Relationship between cellular expression levels of HMGA2 and MDA-MB-231 cell migration. (A) Representative MDA-MB-231 clone with HMGA2 over-expression showing cell distribution in the channel at the beginning and at the end of a 6 h migration experiment in different chemical fields, including the normal medium, a 50 ng/ml EGF uniform field, and different doses of EGF gradient; (B) Comparison of the migration distance of HMGA2 over-expressing MDA-MB-231 and mock cells in different chemical fields (as described in A.); (C) Quantitative analysis of the migration distance as determined for parental MDA-MB-231 with endogenous HMGA2, CRISPR/Cas9 HMGA2 knockout clone and LIN28 inhibitor 1632 induced inhibition of endogenous HMGA2 in MDA-MB-231 cells. Pharmacological inhibition of LIN28, a positive regulator of HMGA2, and genetic knockout of the HMGA2 gene resulted in markedly reduced migratory behavior of MDA-MB-231 breast cancer cells and identified HMGA2 as an important mediator of chemotaxis in triple negative breast cancer cells. The results are presented as the average value±standard error of the mean (SEM). *indicates p<0.05.

FIG. 8. A) Design of the whole 9-unit chip and illustration of a single test unit; B) Representative activated T cell distribution images in the 9-unit device at the beginning and end of 1 h migration experiment in different chemical fields, including a medium control, a 100 ng/mL SDF-1α gradient and a 100 ng/mL SDF-1α uniform field; C) Quantitative migration distance analysis for the experiments in (B).

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

As is known to those of skill in the art, abnormal cell migration and chemotaxis is associated with a wide range of diseases such as autoimmune diseases and cancer metastasis.

As will be apparent to one of skill in the art, measurement of cell migration and/or cell mobility and/or chemotaxis of a sample of specific cell types from an individual, for example, a patient, provides information that can be used in a variety of ways, for example, for diagnosis or alternatively ruling out a particular disease or disorder, or for monitoring treatment efficacy of a particular disease or disorder.

Additionally, cell migration may be measured in the presence of a compound of interest to determine if the compound of interest is a cell migration modulating compound, for example, a compound that could be used to promote wound healing or act as an anti-inflammatory compound or prevent cancer metastasis. As will be appreciated by one of skill in the art, screening a significant number of compounds for their effect on cell mobility is an arduous task, one that is highly dependent on ease of reproducibility of the screening method so that meaningful comparisons can be done and that typically requires significant and specialized knowledge and experience by the end user, for designing and/or carrying out the experiment and for interpreting the results.

As known by those of skill in the art, in vivo, cell migration is highly dependent on the type of cell and the chemo-attractant. When measured or monitored ex vivo, cell migration can be affected by many different experimental and/or design parameters and/or assay conditions, including but by no means limited to chemotactic or chemical gradient channel dimensions, barrier dimensions, type of cell binding agent and concentration thereof and chemo-attractant type and concentration thereof. As a consequence, utility of cell migration assays can be limited without reproducible and predictable methods and devices for carrying out these methods. Furthermore, traditional cell migration methods have been difficult to adapt for simultaneous analysis of multiple samples.

Accordingly, described herein is a method for the preparation of one or more microfluidic chemotactic device prototypes, as well as a method for producing a plurality of microfluidic chemotactic devices for use in measuring cell mobility as well as conditions for the reproducible use of same wherein channel and/or barrier dimensions and chemo-attractant and/or cell binding agent concentration and/or cell binding agent type are varied for developing a microfluidic chemotactic device for a particular cell type and chemo-attractant type as well as instructions for use of same in a cell mobility assay. As discussed herein, this process may also require determination of density of applied cells and cell solution volume or sample volume.

Once the parameters have been determined, a production run can be carried out with the same parameters, thereby producing a plurality of microfluidic chemotactic devices and methods and instructions for the use thereof for reliable and reproducible use in cell mobility assays. Specifically, as a result of this process, detailed operation protocols are provided for the end users to follow so that only knowledge of how to use a pipette and a microscope is sufficient to be able to carry out the cell mobility assays as described herein.

As used herein, “optimized” does not necessarily mean the absolute “best” version but merely a version of the device in which “suitable” or “desirable” conditions have been attained that produce reliable and reproducible results.

Specifically, optimization of the reaction conditions for a particular type of sample and/or a particular type of analysis removes the need for additional experimentation by subsequent users and as such does not require that the assays be carried out by highly skilled and/or highly trained individuals. This also allows for comparison of results obtained on different days and/or by different researchers and/or by different research groups, thereby facilitating comparisons and allowing for greater confidence in results.

As will be appreciated by one of skill in the art, described herein is not a single optimized method but rather a process for optimizing or determining assay conditions for reproducible and reliable cell mobility analysis of a wide variety of cell types and chemoattractants.

As will be appreciated by one of skill in the art, the process of optimization requires the systematic modification of experimental conditions, as discussed herein, and measurement of the resulting cell migration data and parameters.

In some embodiments, the migration data and parameters are extracted from time-lapse images taken of the chemotaxis assay. In some embodiments, time-lapse imaging may comprise imaging at least 6 frames per minute using a microscope with an environmental control chamber to maintain the temperature at approximately body temperature, for example, at approximately 37 C. For longer experiments with certain cell types, pH and humidity control may also be required, which can be accomplished by injecting humidified CO₂ mixed with background air.

As discussed herein, individual cells from the time-lapse images can then be tracked over the course of time-lapse images to calculate quantitative cell migration parameters, including but by no means limited to chemotactic index, cell speed, flowtaxis, entropy analysis, angle of migration direction, directionality, pause number, onset time and velocity, various combinations of which can be used for quantification of altered cell migratory behavior, as discussed herein.

As will be appreciated by one of skill in the art, differences in cell migration between sample cells and controls may only be reproducibly detected under certain specific experimental conditions, as discussed herein. Furthermore, selected binding agents and chemotactic agents do not always work as predicted or expected, making testing and optimization of chemotactic experiment parameters, that is, assay conditions essential for reproducibility, that is, reproducible and/or reliable results.

As discussed herein, any suitable cell type of interest may be used, for example, but by no means limited to leukocyte subsets, cancer cells and stem cells.

As will be appreciated by one of skill in the art, the cell type selected will influence the selection of the chemotactic agent and the cell binding agent, as discussed herein.

Herein, the components of the microfluidic chemotaxis device prototype are described in singular, specifically, a chemical gradient generator, a chemical gradient channel, a cell docking structure, and a barrier. It is however to be understood that each microfluidic chemotaxis device prototype may comprise more than one set of each, as discussed herein, so that multiple parameters can be modified and/or tested.

The microfluidic chemotaxis prototype device may comprise:

a chemical gradient generator;

a chemical gradient channel in fluid communication with the chemical gradient generator, said chemical gradient channel arranged to be coated or for coating with a cell binding agent;

a cell docking area for receiving a quantity of cells, said cell docking area separated from said chemical gradient channel by a gap channel that is smaller than the average height of a respective one cell of the quantity of cells, said gap channel being formed by a cell barrier extending across a length of the cell docking area between the cell docking area and the chemical gradient channel;

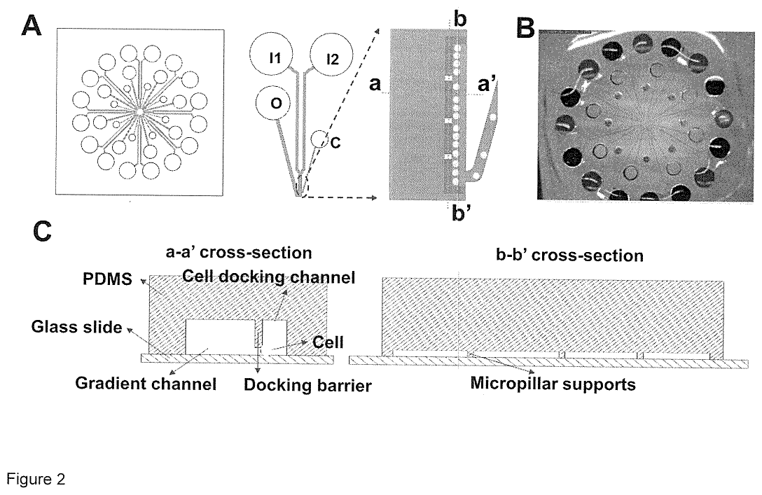

micropillars connected from a top of the gap channel to a glass slide, said glass slide for sealing the microfluidic chemotaxis device, said micropillars supporting the gap channel. Specifically, as shown in FIG. 2C, the micropillars connect the glass slide and the barrier to prevent the collapse of the barrier towards the glass slide during use. As discussed herein, if the barrier collapses, the connection between the chemical gradient channel and the gap channel will be disrupted and no cells will be able to exit the docking area and/or enter the gap channel. In some embodiments, the optimized microfluidic chemotaxis devices prepared by production run or in mass production of the more rigid material may also include micropillars.

As will be appreciated by one of skill in the art and as discussed herein, the prototypes are made of flexible materials, such as for example but by no means limited to PDMS. Other suitable materials will be obvious to one of skill in the art and are within the scope of the invention.

Because of the flexibility of the prototype, a specific amount of pressure must be applied to achieve good sealing between the PDMS structure and the glass slide. If insufficient pressure is applied, a seal is not attained; however, if too much pressure is applied, the cell docking barrier can collapse into the gap channel, thereby ruining the device. Specifically, when a collapse occurs, the cell docking area is disconnected from the gradient channel which means that cells cannot be loaded into the docking area because there is no flow, and the device is ruined. In the experience of the inventors, an inexperienced technician will fail to achieve a proper seal the vast majority of the time and will often destroy the device, meaning that fabrication must be repeated. However, the addition of the columns or micropillars removes this skill requirement and results in a successful sealing rate of nearly 100%. This is important because the fabrication of PDMS devices from a mold will take 2-3 hours, most of which is the baking time for the PDMS to solidify.

As discussed herein, the cell docking area accepts or receives the quantity of cells or the cells of the sample and mechanically confines these cells to the docking area without requiring firm cell adhesion to the substrate. Specifically, the individual cells of the sample are confined to the cell docking area by the barrier, which is smaller than the “height” of the cells so that the cells cannot enter the gap channel and leak into the chemical gradient channel. However, once the chemo-attractant gradient is applied, these cells are able to deform and can pass beneath the cell barrier, thereby entering the gap channel and subsequently the chemical gradient channel, as discussed herein.

As will be appreciated by one of skill in the art, sufficient numbers of cells must be applied to the chemical gradient channel for the data that is obtained to be statistically significant. However, too many cells may impair chemotaxis under certain experimental conditions as may a sample volume that is too dilute or too concentrated. As such, cell density and cell solution volume represent two additional experimental parameters that must be determined. For example, if too many cells are deposited into the cell docking area, physical contact between the cells may force some of the cells out of the docking area and into the gap channel and/or the gradient channel instead of active directional migration. This is more critical for the adherent cell types, such as cancer cells and tissue cells. In contrast, if too few cells are loaded, there may not be a statistically significant number of cells for statistics analysis.

In some embodiments, the cell docking area is connected to a cell loading structure.

In some embodiments, the cell loading structure has a substantially cone-like shape, with the tip of the cone being attached to the cell docking area so that samples can be applied to the large open area or conical portion of the cone, thereby directing the cells of the sample into the cell docking area. That is, the conical portion of the cone may be arranged to accommodate the insertion of a pipette tip or attachment of suitable hoses or pumps for supplying cells to the prototype.

As will be appreciated by one of skill in the art, cell size determines the docking barrier height. For example, a docking barrier that is too low may prevent cells from entering into the gap channel while a docking barrier that is too high can't trap cells in the cell docking area effectively.

As discussed herein, docking barriers for smaller cells, such as immune cells, are typically 2-4 μm while docking barriers for larger cells, such as non-immune cells, for example, cancer cells and tissue cells, are typically 5-10 μm.

Accordingly, as discussed herein, prototypes may be constructed in which chemotaxis of immune cells to a particular attractant is being optimized by testing two or more heights of a docking barrier associated with a particular chemical gradient channel selected from the group consisting of about 2 μm, about 3 μm and about 4 μm.

Similarly, when developing a prototype and associated protocol for chemotaxis of non-immune cells, the height of the docking barrier associated with a particular chemical gradient channel may be tested at two or more values selected from the group consisting of about 5 μm, about 6 μm, about 7 μm, about 8 μm, about 9 μm and about 10 μm.

As used herein in regards the height of the docking barrier, it is to be understood that for example, “5 μm” refers to any value between 4.5 μm and 5.5 μm.

As discussed herein, the inventors have found that a 1 μm difference in barrier height/gap channel depth can make a significant difference for a migration experiment; furthermore, the fabrication technique described herein can vary the height of the barrier with 1 μm accuracy.

As is known to those of skill in the art, for chemotaxis assays, it is essential that the chemical gradient of the chemo-attractant be stable and have flow characteristics that do not bias the direction of migration of the cells. Furthermore, small disturbances can easily distort a gradient that exists in a small-volume microfluidic channel. Finally, it is also important that the gradient remain stable for a predetermined time, for example, for at least the expected duration of the experiment.

The inventors have developed methods for the preparation of stable gradients. In one embodiment two input channels are used which allows for the rapid generation of a chemical gradient in the chemical gradient channel in a pump-free manner.

The properties of the chemical gradient can be characterized by either computer simulation or experimentation. The concentration at the barrier end depends on the flow rate, the diffusion properties of the chemoattractant and the chemical gradient channel dimensions. Furthermore, the position along the chemical gradient channel selected for imaging may also be important in some embodiments. Typically, the concentration at the end of the chemical gradient channel proximal to the gap channel will be 10-20% of the maximum input concentration.

Gradient formation and stability can be tested by adding a fluorescent dye with a similar molecular weight of the chemoattractant (e.g. FITC-Dextran) to the chemoattractant solution for gradient verification purposes. For example, the molecular weight of FITC-Dextran ranges from 4000 to 150,000 Dalton. Because of this, it is possible to choose the one with the closest molecular weight to the chemoattractant. For example, we chose 10KD FITC-Dextran to assess the 8KD IL-8 gradient. Specifically, the differences in fluorescence produced by the dye within the gradient acts as a visual proxy for the chemoattractant by virtue of the similarity in molecular weight.

As discussed herein, the chemoattractant selected depends on the type of cell being tested. For example, IL-8 and fMLP are well-known chemoattractant for human neutrophils. Similarly, SDF-1 alpha is a well-known chemoattractant for lymphocytes. Other suitable chemoattractants will be readily apparent to one of skill in the art.

Similarly, one of skill in the art will have an expectation of what represents a range of suitable possible concentrations for a particular chemoattractant, this needs to be confirmed by experimentation. For example, for chemokines, the range of concentrations is typically in the order of a few nM to a few hundred nMs, that is, from for example 1 nM to 400 nM. Accordingly, in some embodiments of the invention, the optimized chemoattractant concentration may be determined by selecting two or more concentrations from within a range of suitable concentrations and determining which concentration produces the most desirable results, that is, produces a statistically significant, reproducible difference in chemotaxis or cell mobility between a sample of interest and a control.

As will be appreciated by one of skill in the art, the depth and width of the chemical gradient channel also affect cell migration/chemotaxis in a cell type/chemoattractant/binding agent specific manner.

For example, the width of a chemical gradient channel may be on the order of a few hundred micrometers, for example, 100-400 μm, so as to allow significant cell movement across the chemical gradient channel in response to exposure to the chemical gradient or chemo-attractant gradient.

The depth of a chemical gradient channel may be typically 20-100 μm so the presence of cells does not significant affect gradient generation.

The length of the chemical gradient channel is typically in the order of 10 mm to allow enough total channel space for imaging of the cells within the sample and to contribute to the total fluidic resistance, which affects flow rate. Other suitable lengths may also be used.

As such, in some embodiments, the width and depth of the chemical gradient channel for a given cell type/chemoattractant combination may be determined by making two or more microfluidic chemotactic assay units/devices wherein each respective one has a different chemical gradient channel width selected from within the range of 100-400 μm and/or a different depths selected from within the range of 20-100 μm. As will be appreciated by one of skill in the art, these may be varied separately or in combination during the optimization process.

Suitable binding agents will be readily apparent to those of skill in the art. For illustrative purposes, two binding agents—collagen and fibronectin—are discussed herein.

For the cell binding agent, the concentration is important. Specifically, cells will not adhere to the substrate (the top or surface of the chemical gradient channel) if the coating or binding agent concentration is too low. However, if the coating or binding agent concentration is too high, cells will stick to the substrate and will not migrate. The inventors have found that too high of a concentration can be identified as cells undergoing significant polarization trying to move but unable to effectively move away from the spot of initial adherence.

Furthermore, the binding agents do not necessarily work as expected. For example, in a study of skeletal muscle stem cell migration being carried out by the inventors, it was expected that collagen would work better, but it turned out fibronectin worked better. It was expected that because muscle connects to bones through tendon, which is basically collagen, collagen would be a more relevant substrate. However, it has been surprisingly found that skeletal muscle cells like fibronectin perform better in our microfluidic platform.

For fibronectin, the coating concentration may be on the order of mg/mL, for example, within a range of 0.01 mg/mL to 1.0 mg/mL. Accordingly, the fibronectin concentration may be optimized by testing two or more concentrations within the range of 0.01 mg/mL to 1.0 mg/mL.

For collagen, the suitable coating concentration is typically in the order of μg/mL, for example within a range of 0.01 μg/mL to 10 μg/mL. Accordingly, the collagen concentration may be optimized by testing two or more concentrations within the range of 0.01 μg/mL to 10 μg/mL.

It is of note that the parameters or assay conditions such as the concentration of the chemoattractant gradient, the depth and width of the chemical gradient channel, the concentration of the binding agent, the cell density of the applied sample, the volume of the applied sample and the barrier height may be varied systematically. That is, the initial values selected for testing may span a considerable portion of the recited range. Once those parameters have been tested and evaluated, more narrow ranges may be tested to determine the optimized parameters.

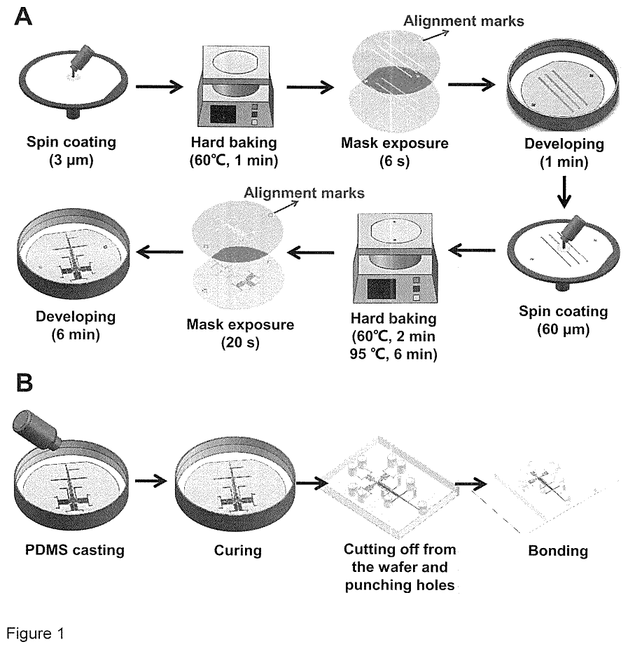

As discussed herein, the prototype of the device may be made of PDMS or another suitable material using a replica molding method, for example, a method as shown in FIG. 1.

It is noted that PDMS is a suitable material for cell migration research because of its air permeability, transparency, bio-compatibility, and low prototyping cost.

As discussed herein, once a prototype with suitable dimensions has been identified, other methods such as hot embossing and injection molding, using plastics materials such as for example but by no means limited to polycarbonate (PC) and polystyrene (PS), are used for a production run, as discussed herein.

As discussed herein, the steps involved in the production of the prototype are:

1) Fabricate a mold on a silicon wafer using photolithography process;

2) Pour liquid PDMS on the mold and bake it to solidify;

3) Peel off PDMS slab from the mold and punch holes for inlets and outlet;

4) Bond the PDMS to a glass slide to seal the channel by plasma treatment.

As will be appreciated by one of skill in the art, the experimental and design parameters and/or dimensions of the device may be determined in stages.

For example, in some embodiments, a first determination may be the gradient channel depth, width, and length, which will confirm that a suitable gradient is being generated. The second determination may be the height of barrier so that the specific height is suitable for the cell type and/or for the chemoattractant. Once these assay parameters are defined, the master for making the PDMS replica is defined. Once this has been done, many PDMS replicas can be made from the same master to determine other parameters or assay conditions such as for example the sample volume, the cell density, the coating molecule identity and concentration thereof and the chemoattractant identity and concentration thereof, as discussed herein. In some cases, it may be that the depth, width and length of the gradient channel may need to be re-determined if suitable assay conditions cannot be determined. Once these experimental parameters have been finalized, a production run of the microfluidic chemotaxis device as described herein can be done so as to produce a plurality of individual microfluidic chemotaxis devices with the same physical parameters, for example, the same barrier height, the same chemical gradient channel depth and the same gradient channel width for use with the determined assay conditions for carrying out reproducible chemotactic assays of the particular cell type of interest, preferably, for the particular cell type of interest and the chemoattractant.

Furthermore, the determination of assay conditions allows for instructions for use of the device to be provided, including the sample volume to be applied, the cell density to be applied, the type and concentration of cell binding agent and the concentration of the chemical gradient and may also include methods for trouble-shooting use of the device. It is noted that the kit may also include aliquots of the cell binding agent and the chemoattractant at the suitable concentrations for application to the microfluidic units/devices together with instructions for the storage and use thereof.

In another aspect of the invention, there is provided a kit comprising a microfluidic chemotaxis device comprising parameters and dimensions as determined via the prototyping process described herein, a quantity of chemoattractant and/or cell binding agent for loading into the channels and instructions for the use thereof.

According to an aspect of the invention, there is provided a method of determining design parameters and experimental conditions or assay conditions for a microfluidic cell mobility assay for a particular cell type of interest, said method comprising providing a microfluidic device comprising: a chemical gradient generator; a chemical gradient channel in fluid communication with the chemical gradient generator, said chemical gradient channel arranged to be coated or for coating with a cell binding agent; a cell docking area for receiving a quantity of cells, said cell docking area separated from said chemical gradient channel by a gap channel that is smaller than the average height of a respective one cell of the quantity of cells, said gap channel being formed by a barrier separating the cell docking area and the chemical gradient channel; and micropillars connected from a top of the gap channel to a glass slide, said glass slide for sealing the microfluidic chemotaxis device, said micropillars supporting the gap channel formed by the barrier for preventing collapse thereof;

determining depth and width of the chemical gradient channel for generating a suitable, stable gradient of a suitable chemoattractant within the chemical gradient channel;

determining a suitable barrier height for the cell type of interest;

preparing a PDMS master of a microfluidic device comprising the determined chemical gradient channel depth and the determined chemical gradient channel width and the determined barrier height;

preparing a plurality of PDMS replicas from the PDMS master and determining the experimental conditions for the cell mobility assay of the cell type of interest by determining chemotaxis or mobility of a quantity of the cell type of interest in more than one of the PDMS replicas while varying at least one of the following parameters:
(1) cell binding molecule applied to the chemical gradient channel;
(2) concentration of the cell binding molecule applied to the chemical gradient channel;
(3) cell density applied to the cell docking area;
(4) sample volume applied to the cell docking area; and
(5) concentration of the chemoattractant in the chemical gradient channel;

and comparing the determined mobilities to select the assay conditions for the microfluidic chemotaxis device.

As discussed above, microfluidic chemotactic device is in some instances referred to in the singular which depending on the context may indicate that the device being referred to is for carrying out one mobility or chemotactic assay, which in other instances is referred to as a microfluidic chemotactic assay unit. It is of note that as discussed herein, the inventors have developed what may also be described as a microfluidic chemotactic device that comprises multiple microfluidic chemotactic assay unit, for example, 8 or 9 radially-arranged units.

In some embodiments, once the experimental conditions are determined, preparing a optimized of finalized microfluidic device or assay unit comprising the chemical gradient channel depth and the determined chemical gradient channel width and the determined barrier height for use with the cell type of interest.

In some embodiments, the optimized or finalized microfluidic devices or assay units are composed of a biocompatible thermoplastic material.

In some embodiments, the finalized or optimized microfluidic devices or assay units are composed of a polycarbonate or a polystyrene material.

According to another aspect of the invention, there is provided a finalized or optimized microfluidic chemotaxis device or assay unit or a plurality of finalized or optimized microfluidic devices or assay units comprising parameters and/or reproducible assay conditions determined according to the above-recited method.

According to another aspect of the invention, there is provided a kit comprising a microfluidic device comprising parameters determined according to the above-recited method and instructions for the use thereof.

In some embodiments, the chemical gradient channel of the microfluidic device comprises the cell binding agent at the determined concentration.

In some embodiments, a kit comprising: at least one finalized or optimized microfluidic device or assay unit comprising the determined chemical gradient channel depth and the determined chemical gradient channel depth and the determined barrier height; and instructions for the use of the microfluidic device reciting the determined cell binding agent concentration for application to the chemical gradient channel; the determined cell density and sample size for application to the cell docking area; and the determined concentration of the chemoattractant in the chemical gradient channel is prepared.

In some embodiments, the kit further comprises a quantity of the chemoattractant at a suitable concentration for preparing the chemical gradient.

In some embodiments, the kit further comprises a quantity of the cell binding agent at the determined concentration for application to the chemical gradient channel.

As discussed herein, the barrier depends on the type of cell of interest.

For example, if the cell type of interest is an immune cell, the barrier may be tested at 2 μm, 3 μm and/or 4 μm.

If the cell type of interest is a non-immune cell, the barrier may be tested at 5 μm, 6 μm, 7 μm, 8 μm, 9 μm and/or 10 μm.

As will be apparent to one of skill in the art, the barrier height is tested by determining if the barrier height is low enough to prevent movement of a non-stimulated cell into the gap channel but high enough to permit movement of a stimulated cell into the gap channel.

The depth and width of the chemical gradient channel may be determined experimentally or by computer simulation.

In some embodiments, gradient formation and stability is tested or confirmed by adding a fluorescent dye with a similar molecular weight to the chemoattractant in the chemical gradient channel, thereby visualizing gradient concentration and stability along the length of the chemical gradient channel.

In some embodiments, the width of the chemical gradient channel for a given cell type/chemoattractant combination is optimized by testing two or more widths from within the range of 100-400 μm. In some embodiments, the depth of the chemical gradient channel for a given cell type/chemoattractant combination is optimized by testing two or more depths from within the range of 20-100 μm.

The cell binding agent may be selected from any suitable cell binding agent known in the art as discussed herein, including but by no means limited to fibronectin and collagen.

For fibronectin, the coating concentration may be on the order of mg/mL, for example, within a range of 0.01 mg/mL to 1.0 mg/mL. Accordingly, the fibronectin concentration may be optimized by testing two or more concentrations within the range of 0.01 mg/mL to 1.0 mg/mL.

For collagen, the suitable coating concentration is typically in the order of μg/mL, for example within a range of 0.01 μg/mL to 10 μg/mL. Accordingly, the collagen concentration may be optimized by testing two or more concentrations within the range of 0.01 μg/mL to 10 μg/mL.

In some embodiments, the experimental parameters or assay conditions are varied by carrying out one or more of the following steps:

(1) selecting a cell binding molecule selected from the group consisting of fibronectin and collagen to for binding to the chemical gradient channel;

(2) if the cell binding molecule is collagen, applying the collagen at a concentration between 0.01 μg/ml and 10 μg/ml to the chemical gradient channel; if the cell binding molecule is fibronectin, applying the fibronectin at a concentration between 0.01 mg/ml and 10 mg/ml to the chemical gradient channel;

(3) applying a quantity of the cell type of interest to the cell docking area at a density selected from 1 million/mL to 10 million/mL;

(4) applying the quantity of the cell type of interest in a sample volume selected from 5 μL to 20 μL; and

(5) applying the chemoattractant to the chemical gradient channel at a concentration selected from 1 nM to 400 nM; to a respective one of the PDMS replicas and comparing cell mobilities under said varied conditions.

In some embodiments, the determined parameters are selected based on the comparison of the cell mobilities under the varied conditions.

In some embodiments, the migration parameters are extracted by taking a series of time-lapsed images of the cells during the chemotaxis assay; tracking mobility of respective ones of the cells using said images, thereby providing mobility data; and calculating mobility of respective ones of the cells from said mobility data.

Wherein the chemotactic assay is started by:

coating the chemical gradient channel with the cell binding agent;

loading a quantity of cells of the cell type of interest to the cell docking area such that said unstimulated cells are prevented from entering the chemical gradient channel by the barrier;

applying the chemotactic agent to the chemical gradient channel; and

sealing the chemotactic device by applying pressure to the glass slide, said micropillars supporting the gap channel formed by the barrier during sealing, thereby preventing collapse thereof of the gap channel during bonding of the PDMS to the glass slide.

As will be apparent to one of skill in the art, sealing the chemotactic device effectively starts the chemotactic assay.

In some embodiments, determining a suitable barrier height for the cell type of interest by preparing at least one microfluidic chemotactic device or assay unit prototype comprising a barrier having a height of between 2-10 μm;

coating the chemical gradient channel of the prototype with the cell binding agent;

loading a quantity of cells of the cell type of interest to the cell docking area;

applying a chemotactic agent at a suitable concentration to stimulate the cells to the chemical gradient channel;

sealing the chemotactic device by applying pressure to the glass slide, said supporting the gap channel formed by the barrier for preventing collapse thereof; and

determining if the barrier height prevents unstimulated cells from entering the chemical gradient channel and allows stimulated cells to enter the chemical gradient channel.

In these embodiments, a negative control, that is, a concentration of the chemotactic agent that is known to be insufficient to induce mobility, for example, a blank or control gradient that does not contain any of the chemotactic agent is used.

In some embodiments, the width of the chemical gradient channel for a given cell type/chemoattractant combination is determined by preparing more than one microfluidic chemotactic device or assay unit having a width selected from within the range of 100-400 μm; and determining chemical gradient properties.

In some embodiments, the depth of the chemical gradient channel for a given cell type/chemoattractant combination is determined by preparing more than one microfluidic chemotactic device or assay unit having a depth selected from within the range of 20-100 μm; and determining chemical gradient properties.

The gradient properties may be characterized by mixing a quantity of the chemoattractant at a suitable concentration with a fluorescent dye having a molecular weight similar to the chemoattractant; loading the mixture onto the chemical gradient channel of the prototype; and monitoring fluorescence over time.

In some embodiments, the width and depth are determined by comparing the gradient properties from the tested widths and depths.

Also described herein are microfluidic devices with radially arranged channel design or radially arranged assay units which allows for multiple simultaneous chemotaxis, for example, for tests of different cell types and/or different gradient conditions and/or different sample. These radially arranged microfluidic devices are capable of stand-alone stable gradient generation using passive pumping and pressure-balancing strategies. One device was validated by testing the migration of fast-migrating human neutrophils and two slower-migrating human breast cancer cell lines, MDA-MB-231 and MCF-7 cells, as discussed below. Furthermore, this radially arranged microfluidic device was useful in studying the influence of the nuclear chromatin binding protein High Mobility Group A2 (HMGA2) on the migration of the human triple negative breast cancer cell line MDA-MB-231, as discussed below.

For example, we validated that MDA-MB-231 breast cancer cells showed optimal and comparable migration in 200, 100, and 50 ng/ml of EGF gradient, while their directional migration decreased significantly when the EGF concentration decreases to 10 ng/ml. This demonstrates that the chemoattractant concentration can't be too high, as too high a concentration will saturate the receptors across the entire cell body, which results in no directional signals for cells. in a situation such as this, specifically, a high concentration gradient area, cell migration can in fact reverse. (Tharp, William G., et al. “Neutrophil chemorepulsion in defined interleukin-8 gradients in vitro and in vivo.” Journal of leukocyte biology 79.3 (2006): 539-554.)

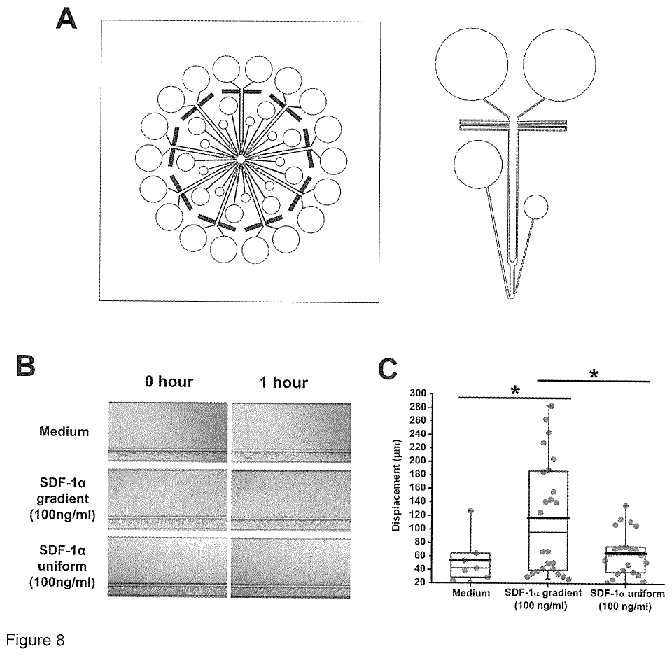

For example, a paper by Ren et al. (Ren et al., 2019, Ann N.Y. Acad. Sci. 1445: 52-61) demonstrates the utility of such a device for the examination of the effect of COPD sputum on activated human peripheral blood T cell migration and chemotaxis. As discussed herein, because of the microfluidic device of the invention, it was possible to carry out these experiments under well-controlled gradient conditions, facilitating the analysis of the complex involvement of T cell trafficking in COPD. FIG. 8B shows representative activated T cell distribution images in the 9-unit device (shown in FIG. 8A) at the beginning and end of 1 h migration experiment in different chemical fields, including a medium control, a 100 ng/mL SDF-1α gradient and a 100 ng/mL SDF-1α uniform field. FIG. 8C shows quantitative migration distance analysis for the experiments in FIG. 8B.

The invention will now be further explained and/or elucidated by way of examples; however, the invention is not necessarily limited to the examples.

### EXAMPLE 1—Design and Fabrication of the Microfluidic Device

The device pattern was designed using AUTOCAD and printed on a transparent film at high resolution. The SU-8 device master was fabricated on a 3-inch silicon wafer by a two-layer photolithography (10). The first layer defines the cell docking structure that is used to align the cells to one side of the gradient channel prior to migration (FIG. 2). Its thickness should be cell specific, with it being slightly lower than the cell size but not be too low to prevent the cells from crossing, as described herein. We chose ˜2.5 μm for neutrophils and ˜7 μm for breast cancer cells. The Polydimethylsiloxane (PDMS) (Sylgard 184; Dow Corning, Midland, USA) device was fabricated using soft-lithography. Inlets (6 mm diameter), outlets (4 mm diameter), and the cell loading port (2 mm diameter) were punched. The PDMS replica was bonded to a glass slide to seal the channels after plasma treatment. The device channels were coated using rat tail collagen type I (3.8 μg/mL; Advanced BioMatrix, San Diego, USA) for one hour and then blocked by 0.4% BSA in RPMI medium for another hour before the migration experiment.

EXAMPLE 2—Design of the Radial Microfluidic Device and Oil-Based Pressure Balancing Strategy

The radially arranged microfluidic device consists of eight identical gradient units (FIG. 2A-B). A 9-member unit is shown in FIG. 8A, as discussed above. Each gradient unit has two chemical inlets merging in a gradient channel, one cell loading inlet, and one waste outlet. The cell docking function was enabled by a barrier to separate the higher cell loading channel and the main gradient channel, thereby initially trapping cells along one side of the gradient channel as previously described (FIG. 2C) (10). Additional support micro-pillars below the barrier added structural device stability during the bonding process between PDMS and the glass slide (FIG. 2C), as discussed below.

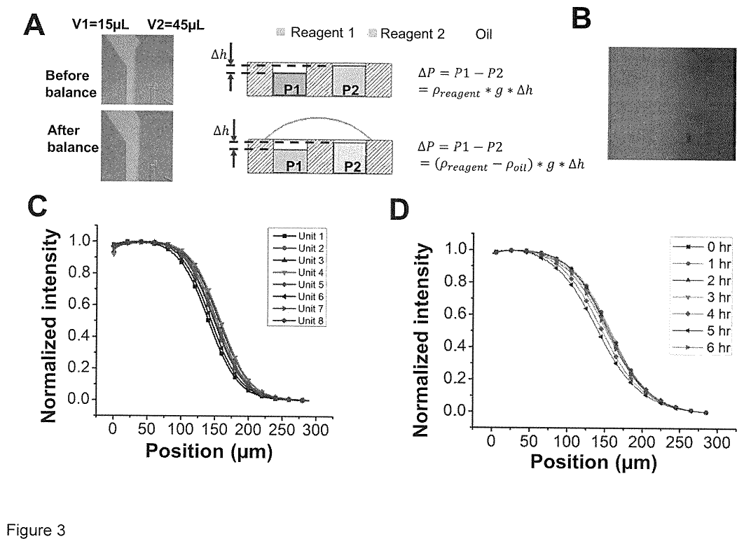

The fluid levels h1 and h2 in the two chemical inlets relative to the waste outlet generated the pressures P1 and P2, which determined the profile and stability of the chemoattractant gradient in the gradient channel (FIG. 3A). At stable equal pressure in the two chemical inlets (P1=P2), the chemoattractant and the buffer streams initially met at the mid-line of the gradient channel and then mixed along the length of the channel to generate a stable chemical gradient across the width of the channel. Variations in loading volumes and dimensions of the inlet wells could easily result in unequal fluid and pressure levels. To address this issue, we applied an oil-based pressure balancing strategy by covering the cell culture media in both chemical inlets with oil (FIG. 3A). We chose silicone oil which is immiscible with the reagents and has a slightly lower density than culture medium, although any suitable oil having a suitable density so as to remain on the surface of the desired reagent may be used, as will be readily apparent to one of skill in the art. The oil on the top of the cell culture media effectively balanced the pressure in the two chemical inlets and this ensured the formation of a stable gradient (FIG. 3A). The oil layer also prevented evaporation of medium during the experiments. This created more stable gradients and allowed for longer observation times. As will be appreciated by one of skill in the art and as demonstrated herein, this is critical because, for certain cell types, the duration of the experiment can be significant, for example, up to 6 hours, as discussed herein, demonstrating that long-term gradient stability is an important improvement in this embodiment of the invention.

According to another embodiment of the invention, there is provided a microfluidic device comprising:

two or more chemotaxis assay units, each respective one chemotaxis units comprising:
a chemical gradient generator comprising a first reagent inlet in fluid communication with a first reagent channel and a second reagent inlet in fluid communication with a second reagent channel, said first reagent inlet and said second reagent inlet arranged to be proximal to one another, said first reagent channel and second reagent channel meeting at a junction to form a gradient channel;
said gradient channel terminating at a cell docking area, said cell docking area being distal to the junction, said cell docking area in fluid communication with a cell inlet for loading cells into the cell docking area, said cell docking area being separated from the gradient channel by a gap channel, said gap channel being arranged to prevent movement of cells from the cell docking area into the gradient channel prior to chemotaxis; and
micropillars connected to a top of the gap channel to a glass slide, said glass slide for sealing the chemotaxis assay unit,

wherein the gradient channel of a first respective chemotaxis assay unit is arranged to be proximal to the gradient channel of a second respective chemotaxis unit.

In some embodiments, the first reagent inlet and the second reagent inlet for a given chemotaxis assay unit are sufficiently close to one another to be covered by a single drop of oil, for example, a 30 ul drop, although larger drops may be used. Alternatively, the first reagent inlet and the second reagent inlet may be at least or about 1-2 mm apart, that is, sufficiently close yet far enough apart that each inlet can be accessed and loaded individually and separately without cross-contamination.

In some embodiments, there may be two or more, two, three or more, three, four or more, four, five or more, five, six or more, six, seven or more, seven, eight or more, eight, nine or more, nine, ten or more, ten, eleven or more, eleven, twelve or more, twelve, thirteen or more, thirteen, fourteen or more or fourteen chemotaxis assay units per microfluidic device. As discussed herein, the ability to carry out multiple assays on one device not only increases throughput but also improves the accuracy of the results because of the elimination of device-to-device variation. Specifically, multiple assays can be carried out under identical (simultaneous) environmental conditions.

As discussed above, the individual chemotaxis assay units are arranged on the microfluidic device such that the gradient channels of adjacent chemotaxis assay units are proximal to one another. As will be appreciated by one of skill in the art, the field of view of a microscope is of a limited size, meaning that, in instances wherein there are higher numbers of chemotaxis units on one microfluidic device, for example, more than three, more than four or more than five, not all of the gradient channels can be viewed at once or at one time. While a mechanized or motorized stage can be used to move between gradient channels, it is desirable to have as short a moving time as possible so that fast time-lapse imaging is possible. Furthermore, having all gradient channels in close proximity to one another will reduce the focus variations between different units.

For example, possible arrangements are shown in FIGS. 2 and 8. As can be seen, in these embodiments, the respective chemotaxis assay units are arranged radially around a common center, which may be proximal to a central or center region of the microfluidic device. That is, the individual gradient channels are arranged radially around a common center so that the stage of the microscope can be moved so that each respective gradient channel can be viewed and photographed while the stage is moved a minimal distance.

Referring again to FIGS. 2 and 8, as can be seen, each respective chemotaxis assay unit is arranged so that there is a relatively small imaging region that includes all of the gradient channels such that the respective gradient channels are proximal to one another while the reservoirs are on the outside or edges of the imaging regions.

### EXAMPLE 3—Cells and Reagents Preparation

Interleukin-8 (IL8) (R&D systems, Minneapolis, USA), N-Formylmethionyl-leucyl-phenylalanine (fMLP) (Sigma, Oakville, Canada) and epidermal growth factor (EGF) (Bachem, Torrance, USA) were used as the chemoattractant for neutrophils and breast cancer cells, respectively, in the radial microfluidic chip. Neutrophils were isolated from blood of healthy donors using a negative magnetic isolation kit (STEMCELL, Vancouver, Canada). The human breast cancer cell lines MDA-MB-231 and MCF-7 were obtained from ATCC and cultured in DMEM/F12 plus 5% FBS. MDA-MB-231 cells containing a pcDNA3 human full-size HMGA2 expression construct or an empty vector control, as well as HMGA2-knockout MDA-MB-231 cells transduced with a specific lentiviral CRISPR/Cas9 construct (pLV-U6g-EPCG-HMGA2, HS0000278246; Sigma, Oakville, Canada), were cultured with DMEM/F12 medium plus 5% FBS. We employed a rabbit polyclonal anti-HMGA2 antibody (Cell Signalling Technologies, Whitby, Canada) in Western blot analysis to identify MDA-MB-231 clones with homozygous deletion of HMGA2. The small molecule Lin28 inhibitor 1632 (R&D Systems, Minneapolis, USA) was used at 10 and 20 μM to down-regulate endogenous HMGA2 in MDA-MB-231 cells.

### EXAMPLE 4—Microfluidic Chemotaxis Experiment

Two microliters of cell suspension were added to the cell loading port of each unit. When the cells had collected in the docking area, chemoattractant solution and migration medium were added to the corresponding inlets (FIG. 2). We used 100 nM IL-8 and 100 nM fMLP dissolved in RPMI-1640 with 0.4% BSA neutrophils. For the migration studies with the human breast cancer cells, EGF (10 ng/ml, 50 ng/ml, 100 ng/ml, 200 ng/ml) dissolved in DMEM/ F-12 plus 1% FBS served as chemoattractant.

To characterize proper gradient generation, FITC-dextran (10 kD; Sigma, Oakville, Canada) was added to the solution containing the chemoattractant and fluorescent images were captured using an inverted fluorescent microscope (Ti-U; Nikon, Mississauga, Canada). Upon adding the chemoattractant solution, the inlets were covered and connected with silicone oil (A12728-22, density=0.963 g/ml; Alfa Aesar, Tewksbury, USA) to balance the pressure difference between the two inlets (FIG. 3A). The microscope stage was enclosed by an environmental control chamber to maintain the temperature at 37° C. during the cell migration experiments. Time-lapse images (10 sec intervals for neutrophils and 3 min intervals for the breast cancer cells) recorded cell migration. The faster migration of neutrophils was recorded for 20 minutes, whereas migration of the breast cancer cells was monitored for 6 hours.

### EXAMPLE 5—Data Analysis

Cell migration and chemotaxis were quantified by calculating the migration distance the cells moved away from the docking site. For MDA-MB-231 experiments, trajectories of representative cells were tracked by manual tracking method using ImageJ. The migration angle ϕ is defined as the angle of the cell displacement vector in relation to the direction of the gradient and was calculated in 12 min intervals. The cosine of ϕ was used to indicate the directionality of cell migration relative to the gradient: 1 indicates cell migration perfectly along the gradient direction; −1 indicates cell migration along the opposite direction of the gradient; a value between 1 and −1 indicates the level of deviation of cell migration from the gradient direction. The cell shape was outlined using ImageJ. Each condition was repeated in at least three independent experiments. For statistical analysis, the two-sample Student's t-test was used to compare different conditions using OriginPro. P<0.05 was considered statistically significant and indicated by an asterisk.

### EXAMPLE 6—Identical and Stable Gradient Formation in the Radial Microfluidic Device

To verify the gradient uniformity and stability in the eight chemotaxis units, medium containing FITC-dextran and medium alone were loaded into either of the two chemical inlets of the octameric chemotaxis unit and sealed with a layer of silicone oil as described above. We used fluorescence imaging to monitor gradient formation in the gradient channels (FIG. 3B). Our results showed that the gradient profiles in all eight channels were identical (FIG. 3C). In addition, the gradient profile in each channel remained stable over the 6 h observation period for the breast cancer cells, as discussed above.

Thus, the radial microfluidic device can generate identical and stable chemical gradients in each of the eight units for the time required to perform the chemotaxis experiments (FIG. 3D).

### EXAMPLE 7—Chemotaxis of Neutrophils and Cancer Cells in the Radial Microfluidic Device

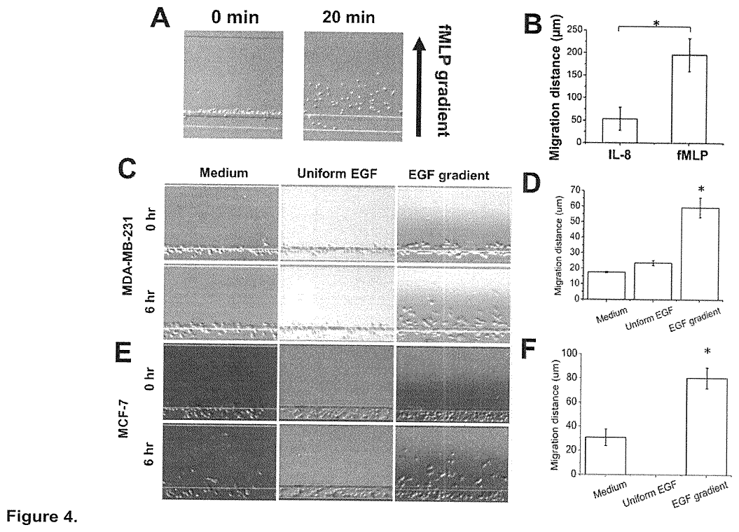

We validated our radial chemotaxis device using isolated human blood neutrophils and two human breast cancer cell lines. Neutrophils were exposed to different chemoattractant gradients in the radial microfluidic device and cell migration was quantified within a 20-minute migration experiment (FIG. 4A). Both fMLP and IL-8 stimulated neutrophil chemotaxis. The current experimental settings resulted in a more than 3-fold stronger chemotaxis response of human neutrophils with the fMLP gradient than the IL-8 gradient (FIG. 4B).

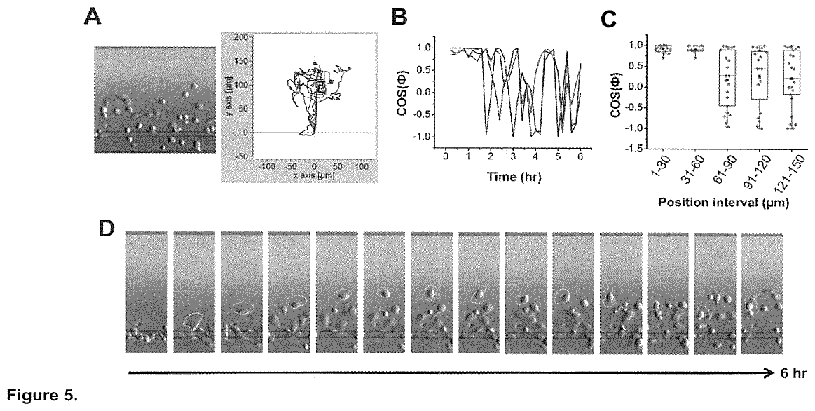

We employed the MDA-MB-231 and MCF-7 cells to test the chemotaxis response of these human breast cancer cell lines in the radial microfluidic device. When exposed to a gradient of epidermal growth factor (EGF; 100 ng/mL), MDA-MB-231 cells actively migrated out of the docking structure towards the EGF gradient. By contrast, MDA-MB-231 cells exposed to a uniform field of EGF (100 ng/mL) or normal culture medium remained stationary in the docking structure and failed to show a chemotaxis response (FIG. 4C-D). Similar results were obtained for MCF-7 cells (FIG. 4E-F). These results confirmed that the new radial microfluidic device was suitable for quantifying directional migration of human breast cancer cells when exposed to an EGF gradient. This device also allowed us to monitor changes in cell morphology and track directionality of cell movement (FIG. 5). When positioned within an area of the gradient with low EGF concentration, the breast cancer cells displayed higher migration directionality and extended lamellipodia towards an area of the gradient with higher EGF concentrations. However, as the tumor cells moved further into the EGF gradient channel, they displayed decreased directionality and more fluctuating lamellipodia orientation (FIG. 5).

### EXAMPLE 8—HMGA2 in Chemotaxis of Breast Cancer Cells using the Radial Microfluidic Device

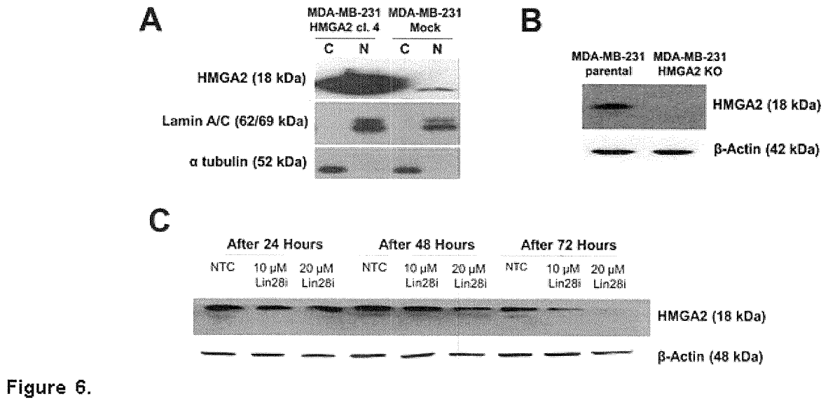

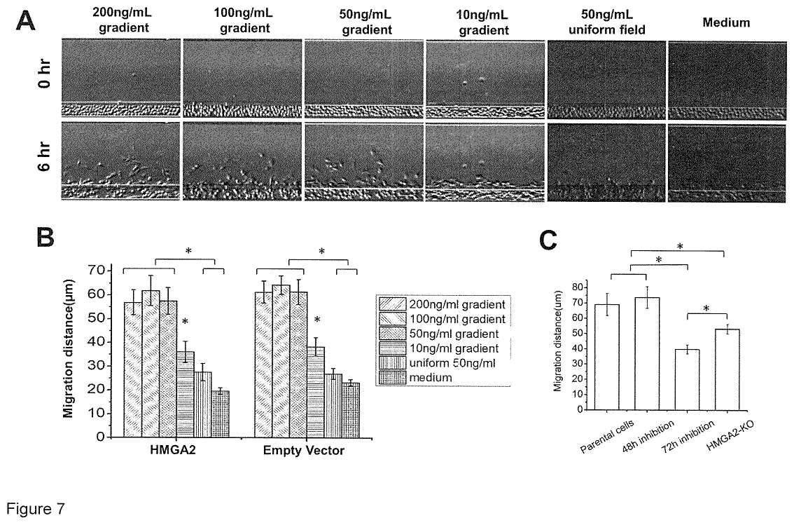

In human breast cancer cells with triple negativity for estrogen receptor, progesterone receptor and HER2, both the fetal oncogene HMGA2 and soluble EGF are frequently up-regulated and are clinical risk factors for increased metastasis^{11, 12}. We have used the radial microfluidic device to determine whether the level of nuclear HMGA2 protein can affect EGF-mediated chemotaxis. The human triple negative breast cancer cell line MDA-MB-231 is an endogenous producer of HMGA2. We generated stable transfectants with over-expression of human full-size HMGA2 as confirmed by Western blot analysis (FIG. 6A). When these transfectants were exposed to different EGF gradients (200, 100, 50 and 10 ng/mL), HMGA2 over-expressing. and mock MDA-MB-231 stable transfectants displayed enhanced and directional migration with all EGF gradients tested, while such migratory behaviour was absent when these cells were exposed to a uniform EGF field (50 ng/mL) or normal medium (FIG. 7A).

Analysis of the migration distance revealed that the EGF gradient generated by the lowest EGF concentration used (10 ng/mL) caused both HMGA2 over-expressing and mock transfectants to migrate the shortest distance from the docking site towards the EGF gradient. However, even this small EGF gradient was sufficient to induce significantly stronger migration when compared to a uniform EGF field (50 ng/mL) or normal medium control (FIG. 7B). Despite the different levels of nuclear HMGA2, both the over-expressing and mock stable transfectants displayed similar migratory responses to the different EGF gradients. This suggested that the endogenous expression level of HMGA2 in MDA-MB-231 was sufficient to elicit a maximal chemotaxis response and excess HMGA2 had no additive effect.

To test this hypothesis and further characterize the role of HMGA2 in chemotaxis of MDA-MB-231, we opted for a pharmacological (LIN28 inhibitor 1632) and CRISPR/Cas9 mediated HMGA2 knockout strategy (FIG. 4B-C). Treatment with the LIN28 inhibitor had been shown to reduce HMGA2 levels (13). Exposure to LIN28 inhibitor for 72 h caused a verifiable reduction of endogenous HMGA2 levels in MDA-MB-231 cells as determined by Western blot analysis (FIG. 6C). This coincided with a significant reduction in chemotaxis response to the EGF gradient as determined by the migration distance in MDA-MB-231 cells with reduced HMGA2 levels (FIG. 7C). Next, we treated MDA-MB-231 with a specific CRISPR/Cas9 lentiviral construct and the homozygous loss of HMGA2 expression in MDA-MB-231 HMGA2 knockout (KO) cells was confirmed by Western blot analysis (FIG. 6C). When exposed to a 100 ng/mL EGF gradient in the radial microfluidic chemotaxis assays, we observed a marked reduction in migration distance in MDA-MB-231 KO clones when compared to parental MDA-MB-231 (FIG. 7C). These results demonstrated a new role for HMGA2 in chemotaxis of human triple negative breast cancer cells and exemplify the utility of the radial microfluidic device to discover, characterize and quantify the role of specific biomolecules in chemotaxis. This novel higher throughput device may be a valuable new tool to excel the search for new therapeutic drugs and small molecules that can mitigate chemotaxis in inflammatory and tumor cells. Specifically, multiple small molecules can be tested at once on gradients that are stable for at least several hours, as discussed herein.

We have introduced novel radial microfluidic gradient devices which can perform up to at least eight parallel cell migration experiments simultaneously with independently controlled gradient configuration and cell type. Each of the units includes a docking structure to align the cells at the low concentration area prior to chemotactic migration. The addition of micropillars increased the structural stability of the device by preventing the barrier channels from collapsing during the bonding process, as discussed herein. This significantly improved the success rate of fabrication. We employed a silicone oil-based pressure balancing strategy to ensure identical and stable gradient formation in each of the eight chemotaxis units. We successfully validated this radial microfluidic platform by demonstrating chemotaxis of human neutrophils in IL-8 and fMLP gradients and human breast cancer cells in an EGF gradient. These chemo attractants have previously been shown to promote chemotaxis in neutrophils and breast cancer cells, respectively (14, 11, 10). A gradient with the input EGF concentration of as little as 10 ng/mL was able to significantly increase directional migration of triple negative MDA-MB-231 breast cancer cells. The radial octameric design of the chemotaxis chip offers the unique versatility of eight simultaneous chemotaxis runs with up to eight different chemo attractants, chemoattractant concentrations, and/or time points with the cell model of choice. This standardized high-throughput device generated comparable data sets at a fraction of the time that were unattainable with traditional single unit chemotaxis systems, for example, because of the elimination of inter-device variability or at least reduction in the severity thereof. The results obtained from these radial microfluidic chips were consistent with previous reports for human neutrophils (15) and human breast cancer cells (16). We noticed that the ability of some cell types, such as neutrophils, to migrate fast can pose a challenge when trying to synchronize the migration in different chemotaxis units. This is due to the time required for loading the chemicals and cells in each unit. However, this technical aspect could be compensated for by individual time monitoring of each unit and the development of a mechanism designed to simultaneously initiate chemical and cell loading in all eight chemotaxis units.

In addition to the ability of the radial microfluidic chip to assess the chemotaxis effect of different chemo attractants on various inflammatory cell types as shown here and previously by others (7), this platform is also a valuable new tool to study chemotaxis behaviour of human cancer cells towards specific chemical gradients. Chemotaxis plays an important role in the metastatic process. The latter signals a severe aggravation of cancer with frequent fatal outcome (17). However, little is currently known about the molecular mechanisms involved in chemotaxis behaviour of tumor cells. In part, this is due to a lack of high-throughput assays to investigate chemotaxis. Our radial microfluidic chips addressed this need as exemplified here using for example the human breast cancer lines MDA-MB-231 and MCF-7 to study their chemotaxis response to stable EGF gradients. The results obtained with the radial microfluidics chip were in agreement with previously reported chemotaxis studies using common one-unit chemotaxis systems, thus validating our exemplary eight unit system (16). Furthermore, the radial microfluidic chip was effective in analyzing the effect on chemotaxis of the LIN28 inhibitor compound 1632 and the downstream LIN28 target and chromatin binding factor HMGA2 in human triple negative breast cancer cells. The stem cell protein LIN28 binds to and inactivates the microRNA Let-7. This prevents Let-7 from binding to Let-7 binding sites located within the 3′UTR of HMGA2 transcripts, thus resulting in enhanced cellular protein levels of HMGA2¹³. The inhibition of LIN28 by a small molecule 1632 and reduction of cellular HMGA2 in MDA-MB-231 cells coincided with markedly reduced chemotaxis. These results show the potential of the radial microfluidic chip to serve as a unique screening tool for the expedient identification of pharmacological compounds and small molecules capable of blocking chemotaxis. The radially arranged microfluidics chip is also a suitable platform to study the role of cellular factors and signalling pathways which impact cell migration and chemotaxis. An example is HMGA2 which is up-regulated in fetal and many cancer cells but silenced in normal adult cells (18). HMGA2 is targeted by several signalling pathways and is an important mediator of mesenchymal transition (EMT) (18). CRISPR/Cas9 targeted knockout of endogenously produced HMGA2 resulted in significantly decreased migration and chemotaxis of MDA-MB-231 in an EGF gradient. The ability to generate quantifiable data sets and track the migratory behaviour of individual cells simultaneously in several different chemical gradients or test cell transfectants with specific mutations in a molecule of interest emphasizes the versatility of the radial microfluidics chip.

In conclusion, our exemplary octameric radial microfluidic chip represents a novel platform that permits the study of cell migration and chemotaxis at higher throughput and may serve as an attractive new discovery tool to quantify the ability of novel drugs to interfere with chemotaxis of inflammatory and/or tumor cells.

The scope of the claims should not be limited, by the preferred embodiments set forth in the examples but should be given the broadest interpretation consistent with the description as a whole.

### REFERENCES

1. Luster, A. D., Alon, R. & von Andrian, U. H. Immune cell migration in inflammation: present and future therapeutic targets. Nat Immunol 6, 1182-1190 (2005)
2. Keller, R. Cell migration during gastrulation. Curr Opin Cell Biol 17, 533-541 (2005)
3. Yamaguchi, H., Wyckoff, J. & Condeelis, J. Cell migration in tumors. Curr Opin Cell Biol 17, 559-564 (2005)
4. Zhao, M. et al. Electrical signals control wound healing through phosphatidylinositol-3-OH kinase-gamma and PTEN. Nature 442, 457-460 (2006)
5. Zigmond, S. Ability of polymorphonuclear leukocytes to orient in gradients of chemotactic factors. J Cell Biol 75, 606-616 (1977)
6. Sackmann, E. K.-H. et al. Characterizing asthma from a drop of blood using neutrophil chemotaxis. Proceedings of the National Academy of Sciences 111, 5813-5818 (2014)
7. Wu, J Wu, X. & Lin, F. Recent developments in microfluidics-based chemotaxis studies. Lab on a Chip 13, 2484-2499 (2013)
8. Agrawal, N., Toner, M. & Irimia, D. Neutrophil migration assay from a drop of blood. Lab on a Chip 8, 2054-2061 (2008)
9. Hamza, B. & Irimia, D. Whole blood human neutrophil trafficking in a microfluidic model of infection and inflammation. Lab on a Chip 15, 2625-2633 (2015)
10. J. Wu, C. Hillier, P. Komenda, R. L. de Faria, S. Santos, D. Levin, M. Zhang and F. Lin, Technology, 2016, 4, 104-109.
11. J. T. Price, T. Tiganis, A. Agarwal, D. Djakiew and E. W. Thompson, Cancer research, 1999, 59, 5475-5478.
12. P. Wend, S. Runke, K. Wend, B. Anchondo, M. Yesayan, M. Jardon, N. Hardie, C. Loddenkemper, I. Ulasov and M. S. Lesniak, EMBO molecular medicine, 2013, 5, 264-279.
13. J. Balzeau, M. R. Menezes, S. Cao and J. P. Hagan, Frontiers in genetics, 2017, 8, 31.
14. N. Li Jeon, H. Baskaran, S. K. W. Dertinger, G. M. Whitesides, L. Van De Water and M. Toner, Nature Biotechnology, 2002, 20, 826.
15. J. Wu, C. Hillier, P. Komenda, R. L. de Faria, D. Levin, M. Zhang and F. Lin, PloS one, 2015, 10, e0126523.
16. S.-J. Wang, W. Saadi, F. Lin, C. M.-C. Nguyen and N. L. Jeon, Experimental cell research, 2004, 300, 180-189.
17. H. Yamaguchi, J. Wyckoff and J. Condeelis, Curr Opin Cell Biol, 2005, 17, 559-564.
18. A. Morishita, M. R. Zaidi, A. Mitoro, D. Sankarasharma, M. Szabolcs, Y. Okada, J. D′Armiento and K. Chada, Cancer research, 2013.

1. A method of optimizing design parameters and/or experimental conditions for a microfluidic cell mobility assay of a particular cell type, said method comprising providing a microfluidic device comprising: a chemical gradient generator; a chemical gradient channel in fluid communication with the chemical gradient generator, said chemical gradient channel arranged to be coated with a cell binding agent; a cell docking area for receiving a quantity of cells, said cell docking area separated from said chemical gradient channel by a gap channel that is smaller than the average height of a respective one cell of the quantity of cells, said gap channel being formed by a barrier separating the cell docking area and the chemical gradient channel; and micropillars connected from a top of the gap channel to a glass slide, said glass slide for sealing the microfluidic chemotaxis device, said micropillars supporting the gap channel for preventing collapse thereof;
determining optimized depth and width of the chemical gradient channel for generating a suitable, stable gradient of a suitable chemoattractant within the chemical gradient channel;
determining a suitable barrier height for the cell type of interest;
preparing a PDMS master of a microfluidic device comprising the optimized chemical gradient channel depth and the optimized chemical gradient channel depth and the optimized barrier height;
preparing a plurality of PDMS replicas from the PDMS master; and
optimizing the experimental conditions for the cell mobility assay of the cell type of interest by determining mobility of the cell type of interest in one of the PDMS replicas while varying at least one of the following parameters:
(1) cell binding molecule applied to the chemical gradient channel;
(2) concentration of the cell binding molecule applied to the chemical gradient channel;
(3) cell density applied to the cell docking area;
(4) sample volume applied to the cell docking area; and
(5) concentration of the chemoattractant in the chemical gradient channel;
and comparing the determined mobilities to select the optimized design parameters for the microfluidic chemotaxis device.

2. The method according to claim 1 wherein once the experimental conditions are optimized, preparing an optimized microfluidic device comprising the optimized chemical gradient channel depth and the optimized chemical gradient channel depth and the optimized barrier height.

3. The method according to claim 1 wherein the optimized microfluidic device is composed of a biocompatible thermoplastic material.

4. The method according to claim 1 wherein the optimized microfluidic device is composed of a polycarbonate or a polystyrene material.

5. The method according to claim 1 wherein cell type of interest is an immune cell and the barrier is tested at 2 μm, 3 μm and/or 4 μm.

6. The method according to claim 1 wherein the cell type of interest is a non-immune cell and the barrier is tested at at least two heights selected from the group consisting of: 5 μm, 6 μm, 7 μm, 8 μm, 9 μm and 10 μm.

7. The method according to claim 1 wherein gradient formation and stability is confirmed by adding a fluorescent dye with a similar molecular weight to the chemoattractant.

8. The method according to claim 1 wherein the width of the chemical gradient channel for a given cell type/chemoattractant combination is optimized by testing two or more widths from within the range of 100-400 μm.

9. The method according to claim 1 wherein the depth of the chemical gradient channel for a given cell type/chemoattractant combination is optimized by testing two or more depths from within the range of 20-100 μm.

10. The method according to claim 1 wherein the cell binding agent is fibronectin and the coating concentration is optimized by testing two or more concentrations within the range of 0.01 mg/mL to 1.0 mg/mL.

11. The method according to claim 1 wherein the cell binding agent is collagen and the suitable coating concentration is optimized by testing two or more concentrations within the range of 0.01 μg/mL to 10 μg/mL.

12. A microfluidic chemotaxis device comprising optimized parameters determined according to the method of claim 1.

13. A kit comprising a microfluidic device comprising optimized parameters according to the method of claim 1 and instructions for the use thereof.

14. The kit according to claim 13 wherein the microfluidic device comprises the optimized chemical gradient channel depth and the optimized chemical gradient channel depth and the optimized barrier height determined according to the method of claim 1; and the instructions recite: the optimized cell binding agent concentration for application to the chemical gradient channel; the optimized cell density and sample size for application to the cell docking area; and the optimized concentration of the chemoattractant in the chemical gradient channel is prepared.

15. The kit according to claim 13 wherein the kit further comprises a quantity of the chemoattractant at a suitable concentration for preparing the optimized chemical gradient.

16. The kit according to claim 13 wherein the kit further comprises a quantity of the cell binding agent at the optimized concentration for application to the chemical gradient channel.

17. The kit according to claim 13 wherein the chemical gradient channel comprises the cell binding agent at the optimized concentration.

18. A microfluidic device comprising:
two or more chemotaxis assay units, each respective one chemotaxis units comprising:
a chemical gradient generator comprising a first reagent inlet in fluid communication with a first reagent channel and a second reagent inlet in fluid communication with a second reagent channel, said first reagent inlet and said second reagent inlet arranged to be sufficiently proximal to one another, said first reagent channel and second reagent channel meeting at a junction to form a gradient channel;
said gradient channel terminating at a cell docking area, said cell docking area being distal to the junction, said cell docking area in fluid communication with a cell inlet for loading cells into the cell docking area, said cell docking area being separated from the gradient channel by a gap channel, said gap channel being arranged to prevent movement of cells from the cell docking area into the gradient channel prior to chemotaxis; and
micropillars connected to a top of the gap channel to a glass slide, said glass slide for sealing the chemotaxis assay unit,
wherein the gradient channel of a first respective chemotaxis assay unit is arranged to be proximal to the gradient channel of a second respective chemotaxis unit.
